(19) United States
(12) Patent Application Publication
(10) Pub. No.: US 2017/0112746 A1
(43) Pub. Date: Apr. 27, 2017

(54) ANTIPERSPIRANT COSMETICS COMPRISING SPECIFIC PROTEINS FROM ADNEXA OF MAMMALS, BIRDS, FISH, INSECTS OR CRUSTACEANS AND INCLUDING NO ALUMINUM AND/OR ZIRCONIUM HALIDES AND/OR HYDROXY HALIDES

(71) Applicant: Henkel AG & Co. KGaA, Duesseldorf (DE)

(72) Inventors: Bernhard Banowski, Duesseldorf (DE); Stefan Evers, Haan (DE)

(73) Assignee: Henkel AG & Co. KGaA, Duesseldorf (DE)

(21) Appl. No.: 15/400,714

(22) Filed: Jan. 6, 2017

Related U.S. Application Data
(63) Continuation of application No. PCT/EP2015/064491, filed on Jun. 25, 2015.

(30) Foreign Application Priority Data
Jul. 8, 2014 (DE) 10 2014 213 226.6

(51) Int. Cl. A61K 8/64; A61K 8/92; A61Q 15/00

(52) U.S. Cl. CPC A61K 8/64; A61Q 15/00; A61K 8/92; A61K 2800/30

(57) ABSTRACT

The present invention relates to an antiperspirant cosmetic agent including at least one specific protein from cutaneous appendages of mammals, birds, fish, insects, or crustaceans and including no aluminum and/or zirconium halides and/or hydroxyhalides. The present invention further relates to the use of a specific protein and to a non-therapeutic method for reducing body perspiration. Adding or using said at least one specific protein ensures that the sweat gland(s) are effectively influenced, thus resulting in a significant reduction in axillary sweat even in the absence of antiperspirant aluminum salts.

### FIELD OF THE INVENTION

The present invention generally relates to an antiperspirant cosmetic agent without aluminum and/or zirconium halides and/or hydroxyhalides, which includes at least one substance selected from the group of cosmetic oils that are liquid at 20° C. and 1,013 hPa, odorous substances, and waxes, optionally at least one propellant, and specific proteins from cutaneous appendages of mammals, birds, fish, insects, or crustaceans. The addition of the at least one specific protein results in an effect on the sweat gland(s).

Furthermore, the present invention relates to a packaging unit (kit of parts), containing a cosmetic agent according to the invention and a cosmetic agent having at least one antiperspirant active substance.

The present invention also relates to the use of specific proteins from cutaneous appendages of mammals, birds, fish, insects, or crustaceans to at least partially affect the sweat gland(s).

In addition, the present invention relates to the use of a combination, including at least one substance selected from the group of cosmetic oils that are liquid at 20° C. and 1,013 hPa, odorous substances, and waxes, optionally at least one propellant, and specific proteins from cutaneous appendages of mammals, birds, fish, insects, or crustaceans, to reduce and/or prevent sweat, particularly axillary sweat or sweat of other body regions. The combination according to the invention includes no aluminum and/or zirconium halides and/or hydroxyhalides.

Furthermore, the present invention relates to an antiperspirant cosmetic agent without aluminum and/or zirconium halides and/or hydroxyhalides, which includes at least one substance selected from the group of cosmetic oils that are liquid at 20° C. and 1,013 hPa, odorous substances, and waxes, optionally at least one propellant, and at least one specific keratin isolated from sheep's wool or goat's wool. The addition of the at least one specific protein results in an effect on the sweat gland(s).

Finally, the present invention relates to a non-therapeutic cosmetic method for preventing and/or reducing the perspiration of the body, wherein an antiperspirant cosmetic agent according to the invention is applied to a skin surface, particularly to the skin of the axillae, and remains on the skin surface for at least 1 hour, preferably for at least 2 hours, more preferably for at least 4 hours, particularly for at least 6 hours.

### BACKGROUND OF THE INVENTION

The washing, cleaning, and care of one's own body is a basic human need, and modern industry is continually attempting to meet these human needs in a variety of ways. Especially important for daily hygiene is the lasting elimination or at least reduction of body odor and axillary moisture. Numerous specific deodorizing or antiperspirant body care agents developed for use in body regions having a high density of sweat glands, particularly in the axillary region, are known in the prior art. Said body care agents are formulated in a wide range of product forms, for example as a powder, stick, aerosol spray, pump spray, liquid and gel roll-on application, cream, gel, and impregnated flexible substrates (deodorant wipes).

Cosmetic antiperspirants of the prior art include, in addition to at least one oil or wax and one odorous substance component or perfume, at least one antiperspirant compound, particularly in the form of aluminum and/or zirconium halides and/or hydroxyhalides. Said antiperspirant compounds reduce the secretion of sweat of the body by temporarily constricting and/or plugging the excretory ducts of the sweat glands so that the amount of sweat can be reduced by approximately 20 to 60 percent. Furthermore, said antiperspirant compounds have an additional deodorizing effect because of the antimicrobial action of said antiperspirant compounds.

Aluminum and/or zirconium halides and/or hydroxyhalides, in conjunction with the acidic pH value of these antiperspirants, can lead to unpleasant skin reactions in some users. Furthermore, the use of the aforementioned antiperspirant compounds can lead to stains on clothing.

Therefore, there is a need for replacing antiperspirant aluminum and/or zirconium halides and/or hydroxyhalides with other antiperspirant cosmetic active substances. Said antiperspirant active substances should have good antiperspirant action and good skin compatibility and should be easy to formulate. Furthermore, said antiperspirant active substances should not have a negative effect on the storage stability of the antiperspirant cosmetic agents.

The present invention addresses the problem of providing an antiperspirant cosmetic agent that avoids or at least lessens the disadvantages of the prior art and that has good skin compatibility and also reliably reduces axillary moisture. Furthermore, the antiperspirant cosmetic agent should have high storage stability.

It has now surprisingly been found that, if at least one protein from cutaneous appendages of mammals, birds, fish, insects, or crustaceans that causes a change in the light absorption of 1 to 100% in the event of a change in the pH value of at least 0.5 in a pH range of pH 4.0 to pH 8.0 is used in cosmetic agents without antiperspirant aluminum and/or zirconium halides and/or hydroxyhalides, the result is antiperspirant action that is nearly comparable to the antiperspirant action of formulations having aluminum salts and/or aluminum-zirconium complexes.

Furthermore, other desirable features and characteristics of the present invention will become apparent from the subsequent detailed description of the invention and the appended claims, taken in conjunction with the accompanying drawings and this background of the invention.

### BRIEF SUMMARY OF THE INVENTION

An antiperspirant cosmetic agent, including at least one substance selected from the group of cosmetic oils that are liquid at 20° C. and 1,013 hPa, odorous substances, and waxes; propellant in a total amount of 0 to 99 wt %, with respect to the total weight of the antiperspirant cosmetic agent; and at least one protein in a total amount of 0.1 to 70 wt %, with respect to the total weight of the antiperspirant cosmetic agent, wherein the at least one protein occurs in cutaneous appendages of mammals, birds, fish, insects, or crustaceans and wherein the at least one protein causes a change in the light absorption of 1 to 100% in the event of a pH value change of at least 0.5 in a pH range of pH 4.0 to pH 8.0, at a temperature of 20° C. to 40° C. and a concentration of the protein of 0.001 to 10 wt %, with respect to the total weight of the sample mixture used to determine the change in the light absorption, wherein the antiperspirant cosmetic agent includes no aluminum and/or zirconium halides and/or hydroxyhalides.

A packaging unit (kit of parts), comprising—formulated separate from each other—at least one first container (C1), containing a cosmetic agent (M1) comprising at least one antiperspirant active substance; and at least one second container (C2), containing a cosmetic agent (M2) comprising at least one protein, wherein the at least one protein occurs in cutaneous appendages of mammals, birds, fish, insects, or crustaceans, wherein the at least one protein causes a change in the light absorption of 1 to 100% in the event of a pH value change of at least 0.5 in a pH range of pH 4.0 to pH 8.0 at a temperature of 20° C. to 40° C. and a concentration of the protein of 0.001 to 10 wt %, with respect to the total weight of the sample mixture used to determine the change in the light absorption, and wherein the cosmetic agent (M2) includes no aluminum and/or zirconium halides and/or hydroxyhalides.

The use of at least one protein to at least partially influence the sweat gland(s), wherein the at least one protein occurs in cutaneous appendages of mammals, birds, fish, insects, or crustaceans and wherein the at least one protein causes a change in the light absorption of 1 to 100% in the event of a pH value change of at least 0.5 in a pH range of pH 4.0 to pH 8.0 at a temperature of 20° C. to 40° C. and a concentration of the protein of 0.001 to 10 wt %, with respect to the total weight of the sample mixture used to determine the change in the light absorption.

The use of a combination, including at least one substance selected from the group of cosmetic oils that are liquid at 20° C. and 1,013 hPa, odorous substances, and waxes; propellant in a total amount of 0 to 99 wt %, with respect to the total weight of the antiperspirant cosmetic agent; and at least one protein in a total amount of 0.1 to 70 wt %, with respect to the total weight of the antiperspirant cosmetic agent, wherein the at least one protein occurs in cutaneous appendages of mammals, birds, fish, insects, or crustaceans, wherein the at least one protein causes a change in the light absorption of 1 to 100% in the event of a pH value change of at least 0.5 in a pH range of pH 4.0 to pH 8.0 at a temperature of 20° C. to 40° C. and a concentration of the protein of 0.001 to 10 wt %, with respect to the total weight of the sample mixture used to determine the change in the light absorption, and wherein the combination includes no aluminum and/or zirconium halides and/or hydroxyhalides, to reduce and/or prevent sweat, particularly axillary sweat or sweat of other body regions.

An antiperspirant cosmetic agent, including at least one substance selected from the group of cosmetic oils that are liquid at 20° C. and 1,013 hPa, odorous substances, and waxes; propellant in a total amount of 0 to 99 wt %, with respect to the total weight of the antiperspirant cosmetic agent; and at least one keratin isolated from sheep's wool or goat's wool in a total amount of 0.1 to 70 wt %, with respect to the total weight of the antiperspirant cosmetic agent, wherein the keratin is hydrolyzed and/or wherein the keratin includes at least one lauryldimonium hydroxypropyl group, wherein the antiperspirant cosmetic agent includes no aluminum and/or zirconium halides and/or hydroxyhalides.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

The subject of the present invention is an antiperspirant cosmetic agent, including
a) at least one substance selected from the group of cosmetic oils that are liquid at 20° C. and 1,013 hPa, odorous substances, and waxes,
b) propellant in a total amount of 0 to 99 wt %, with respect to the total weight of the antiperspirant cosmetic agent, and
c) at least one protein in a total amount of 0.1 to 70 wt %, with respect to the total weight of the antiperspirant cosmetic agent, wherein the at least one protein occurs in cutaneous appendages of mammals, birds, fish, insects, or crustaceans and wherein the at least one protein causes a change in the light absorption of 1 to 100% in the event of a pH value change of at least 0.5 in a pH range of pH 4.0 to pH 8.0, at a temperature of 20° C. to 40° C. and a concentration of the protein of 0.001 to 10 wt %, with respect to the total weight of the sample mixture used to determine the change in the light absorption,
   wherein the antiperspirant cosmetic agent includes no aluminum and/or zirconium halides and/or hydroxyhalides.

The use of the at least one protein from cutaneous appendages of mammals, birds, fish, insects, or crustaceans having the specific physical properties mentioned above in the antiperspirant cosmetic agents according to the invention results in a deliberate effect on the sweat gland(s), though there is no intention of being restricted to this theory. Said deliberate effect on the sweat gland(s) can consist, for example, in gel formation by the at least one protein at pH values that exist only within the excretory ducts of the sweat glands. In this way, effective plugging of the excretory ducts of the sweat glands can be ensured without a reduction in the antiperspirant action of the cosmetic agent according to the invention because of premature undesired gel formation due to the addition of the at least one specific protein. However, the deliberate effect on the sweat gland(s) can also consist in a disturbance of the charge equilibrium within the sweat gland(s), which leads to an effect on the production of sweat, particularly to a reduction in the production of sweat. Therefore, an effective reduction in axillary sweat is ensured even in the absence of antiperspirant aluminum and/or zirconium halides and/or hydroxyhalides.

According to the invention, the term “antiperspirant” is understood to mean the reduction of the perspiration of the sweat glands of the body.

Furthermore, in the context of the present invention, the term “aluminum and/or zirconium halides and/or hydroxyhalides” is understood to mean, in particular, chlorides, bromides, and iodides of aluminum and zirconium and compounds of the formulas Al(OH)_{y}X and Zr(OH)_{z}X, wherein X represents a halide ion in the aforementioned formulas.

Furthermore, in the sense of the present invention, the term “cosmetic oil” is understood to mean an oil that is suitable for cosmetic use and that is not miscible with water in all amounts. The cosmetic oil used according to the invention is neither an odorous substance nor an essential oil.

In addition, in the sense of the present invention, the term “odorous substances” is understood to mean substances that have a molar mass of 74 to 300 g/mol, that include at least one osmophore in the molecule, and that have an odor and/or flavor, i.e., said substances are capable of stimulating the receptors of the hair cells of the olfactory system. Osmophores are groups, in the form of hydroxy groups, formyl groups, oxo groups, alkoxycarbonyl groups, nitrile groups, nitro groups, azide groups, etc., that are covalently bonded to the molecular skeleton. In this context, perfume oils, perfumes, or perfume oil constituents that are liquid at 20° C. and 1,013 hPa also fall under the term “odorous substances” in the sense of the present invention.

Furthermore, in the context of the present invention, the term “waxes” is understood to mean substances that are kneadable or solid to brittle and hard at 20° C., have a coarse to finely crystalline structure, and are colorfully translucent to opaque, but not vitreous. Furthermore, said substances melt above 25° C. without decomposition, flow readily (have low viscosity) slightly above the melting point, have a highly temperature-dependent consistency and solubility, and can be polished under light pressure.

According to the invention, the term “protein” refers to chemical compounds that are condensation products of amino acids, which condensation products are linked by peptide bonds in the manner of an acid amide. The number of amino acids in the proteins is preferably at least 2 and at most 1,000 amino acids. According to the invention, the term “protein” should also be understood to mean hydrolysates of a protein that include protein fractions having different amino acid sequences and molecular weights. Furthermore, in the context of the present invention, this term should also be understood to mean mixtures of proteins that occur in cutaneous appendages.

Furthermore, in the context of the present invention, the term “cutaneous appendages” should be understood to mean all structures that are formed from the dermis and epidermis. This includes, in particular, hair, feathers, horn formations such as horns, finger nails, scutes, claws, and hooves, pearls, scales, armor, and shells.

Furthermore, the term “change in the light absorption of the at least one protein” is understood to mean both the positive and negative change in the light transmittance of the sample mixture, particularly of the protein solution, and the absorption of light by the at least one protein or the sample mixture.

Furthermore, the term “pH value change” is understood to mean continuous change in the pH value. Continuous change in the pH value can be achieved, for example, by the titration, or steady addition, of a base or acid.

According to the invention, the term “sample mixture” refers to a mixture of the at least one specific protein with a solvent, particularly water, buffer, or salt-containing aqueous solutions.

In addition, the term “fatty acids,” as it is used in the context of the present invention, should be understood to mean aliphatic carboxylic acids that have unbranched or branched carbon residues having 4 to 40 carbon atoms. The fatty acids used in the context of the present invention can be naturally occurring fatty acids or synthetically produced fatty acids. Furthermore, the fatty acids can be mono- or polyunsaturated.

Finally, in the context of the present invention, the term “fatty alcohols” is understood to mean aliphatic, monohydric, primary alcohols that have unbranched or branched hydrocarbon residues having 4 to 40 carbon atoms. The fatty alcohols used in the context of the invention can also be mono- or polyunsaturated.

In this document, the specification of wt % relates to the total weight of the antiperspirant cosmetic agents according to the invention, unless otherwise indicated.

As a first constituent a), the cosmetic agents according to the invention include at least one substance selected from the group of cosmetic oils that are liquid at 20° C. and 1,013 hPa, odorous substances, and waxes.

In the context of the present invention, the cosmetic oil that is liquid at 20° C. and 1,013 hPa is selected from the group of (i) volatile cyclic silicone oils, particularly cyclic and linear silicone oils; (ii) volatile non-silicone oils, particularly liquid paraffin oils and isoparaffin oils; (iii) non-volatile silicone oils; (iv) non-volatile non-silicone oils; and (v) mixtures thereof.

According to the invention, the term “volatile oil” refers to oils that have a vapor pressure of 2.66 Pa to 40,000 Pa (0.02 to 300 mm Hg), preferably 10 to 12,000 Pa (0.1 to 90 mm Hg), more preferably 13 to 3,000 Pa (0.1 to 23 mm Hg), particularly 15 to 500 Pa (0.1 to 4 mm Hg), at 20° C. and an ambient pressure 1,013 hPa.

Furthermore, in the sense of the present invention, the term “non-volatile oils” is understood to mean oils that have a vapor pressure of less than 2.66 Pa (0.02 mm Hg) at 20° C. and an ambient pressure of 1,013 hPa.

According to the invention, it can be preferred that mixtures of volatile silicone oils and volatile non-silicone oils are used in the antiperspirant cosmetic agents according to the invention, because a drier skin feel is thereby achieved. Furthermore, it can be preferred in the context of the present invention if the antiperspirant cosmetic agents include a non-volatile silicone oil and/or a non-volatile non-silicone oil in order to mask insoluble constituents, such as talc or ingredients that are dried on the skin.

Especially preferred according to the invention is the use of mixtures of non-volatile and volatile cosmetic oils, because in this way parameters such as skin feel, visibility of the residue, and stability of the antiperspirant cosmetic agent according to the invention can be set and the agent can thus be better adapted to the needs of the consumers.

The volatile and non-volatile silicone oils and volatile and non-volatile non-silicone oils that can be used in the context of the present invention are disclosed, for example, in laid-open applications DE 102010063250 A1 and DE 102012222692 A1.

According to a preferred embodiment of the present invention, the cosmetic oil that is liquid at 20° C. and 1,013 hPa is included in a total amount of 0.02 to 98 wt %, preferably 2 to 85 wt %, preferably 4 to 75 wt %, more preferably 6 to 70 wt %, even more preferably 8 to 60 wt %, particularly 8 to 20 wt %, with respect to the total weight of the antiperspirant cosmetic agent.

At least one odorous substance can also be included as constituent a) of the cosmetic agents according to the invention. However, mixtures of different odorous substances that together produce a pleasant scent are preferably used. Odorous substances that are usable in the context of the present invention are disclosed, for example, in laid-open application DE 102010063250 A1.

Especially pleasant-smelling antiperspirant cosmetic agents according to the invention are obtained if the at least one odorous substance is included in a total amount of 0.00001 to 15 wt %, preferably 0.001 to 9 wt %, more preferably 0.01 to 8 wt %, even more preferably 0.1 to 7 wt %, even more preferably 0.2 to 6 wt %, particularly 0.2 to 2 wt %, with respect to the total weight of the antiperspirant cosmetic agent.

Furthermore, the antiperspirant cosmetic agents according to the invention can include a wax as constituent a). Said wax is preferably selected from the group of (i) fatty acid glycerol mono-, di-, and triesters; (ii) Butyrospermum Parkii (Shea Butter); (iii) esters of saturated, monohydric C₈₋₁₈ alcohols with saturated C₁₂₋₁₈ monocarboxylic acids; (iv) linear, primary C₁₂₋₂₄ alkanols; (v) esters of a saturated, monohydric C₁₆₋₆₀ alkanol and a saturated C₈₋₃₆ monocarboxylic aid; (vi) glycerol triesters of saturated linear C₁₂₋₃₀ carboxylic acids, which can be hydroxylated; (vii) natural plant waxes; (viii) animal waxes; (ix) synthetic waxes; and (x) mixtures thereof. Waxes that can be used with preference in the context of the present invention are disclosed in laid-open application DE 102012222692 A1.

In the context of the present invention, it is preferred if the wax is included in a total amount of 0.01 to 50 wt %, preferably 3 to 40 wt %, more preferably 5 to 30 wt %, particularly 6 to 25 wt %, with respect to the total weight of the antiperspirant cosmetic agent.

According to one embodiment of the present invention, it can be provided that the antiperspirant cosmetic agents according to the invention include a propellant in a total amount of 0 to 99 wt %, with respect to the total weight of the antiperspirant cosmetic agent, as constituent b). If the cosmetic agents according to the invention include a propellant, said propellant is preferably included in a total amount of 1 to 98 wt %, preferably 20 to 90 wt %, more preferably 30 to 85 wt %, particularly 40 to 75 wt %, with respect to the total weight of the antiperspirant cosmetic agent. In this case, the cosmetic agents according to the invention are formulated as propellant-gas-driven aerosols. Preferred propellants (propellant gases) are propane, propene, n-butane, isobutane, isobutylene, n-pentane, pentene, isopentane, isopentene, methane, ethane, dimethyl ether, nitrogen, air, oxygen, nitrous oxide, 1,1,1,3-tetrafluoroethane, heptafluoro-n-propane, perfluoroethane, monochlorodifluoromethane, 1,1-difluoroethane, tetrafluoropropene, individually and in mixtures thereof. Hydrophilic propellant gases, such as carbon dioxide, can also be advantageously used according to the present invention if the proportion of hydrophilic gases is low and lipophilic propellant gas (e.g., propane/butane) is present in excess. Propane, n-butane, isobutane, and mixtures of these propellant gases are especially preferred. It has been found that the use of n-butane as a sole propellant gas can be especially preferred according to the invention.

The antiperspirant cosmetic agent according to the invention includes at least one specific protein from cutaneous appendages of mammals, birds, fish, insects, or crustaceans as a third constituent c).

In the context of the present invention, an especially effective reduction in axillary sweat by means of the at least one specific protein is achieved if the at least one protein is included in a total amount of 0.5 to 60 wt %, preferably 1.0 to 50 wt %, more preferably 1.5 to 40 wt %, even more preferably 2.0 to 30 wt %, particularly 2.0 to 20 wt %, with respect to the total weight of the antiperspirant cosmetic agent. With no intention of being restricted to this theory, the use of the aforementioned amounts of the at least one specific protein results in a significant effect on the sweat gland(s) due to gel formation by the protein in the excretory ducts of the sweat glands or due to an effect on the charge equilibrium within the sweat gland(s). In this way, excellent antiperspirant action is ensured. Furthermore, the use of the aforementioned amounts of the at least one specific protein does not lead to unstable formulations, and therefore the stability of the antiperspirant cosmetic agents according to the invention is ensured even over long time periods of storage.

Especially good results with respect to the reduction of axillary moisture and with respect to skin compatibility and storage stability are obtained if the at least one protein has an average molecular weight Mw of 150 to 100,000 Da, preferably 180 to 50,000 Da, more preferably 200 to 10,000 Da, even more preferably 250 to 8,000 Da, particularly 300 to 5,000 Da. The average molecular weight Mw can be determined by gel permeation chromatography (GPC), for example (Andrews, P.; “Estimation of the Molecular Weights of Proteins by Sephadex Gel-Filtration”; Biochem. J., 1964, 91, pages 222 to 233).

According to a preferred embodiment of the present invention, the at least one protein has an isoelectric point that lies in the range of pH 4.0 to pH 10.0, preferably pH 4.0 to pH 9.5, particularly pH 4.0 to pH 8.0. In particular, proteins that have an isoelectric point in the aforementioned pH range have proven advantageous in the context of the present invention with respect to the antiperspirant action and the stability of the cosmetic agents according to the invention.

Especially high antiperspirant action, skin compatibility, and storage stability are ensured in the context of the present invention if the at least one protein causes a change in the light absorption in the event of a pH value change of at least 0.5 in a pH range of pH 4.5 to pH 7.5, particularly pH 5.0 to pH 7.0, at a concentration of 0.001 to 10 wt % of protein, with respect to the total weight of the sample mixture used for pH measurement, and at a temperature of 20° C. With no intention of being restricted to this theory, the use of the at least one specific protein that causes a change in the light absorption in a certain pH range results in a significantly increased effect on the sweat gland(s) due to pH-selective gel formation in the excretory ducts of the sweat glands or due to disturbance of the charge equilibrium of the sweat gland(s), so that excellent antiperspirant action of the cosmetic agents according to the invention is ensured, which antiperspirant action is comparable to the antiperspirant action of aluminum-salt-containing or aluminum-zirconium-salt-containing cosmetic agents of the prior art.

In the context of the present invention, it is preferred if the pH value change is caused by the addition of hydrogencarbonates or carbonates, particularly sodium hydrogencarbonates.

According to a preferred embodiment of the present invention, the at least one protein is selected from the group of (i) unmodified proteins; (ii) hydrolyzed proteins; (iii) chemically modified proteins, particularly hydrophobically and/or cationically and/or anionically modified proteins; (iv) physically modified proteins, particularly fractionated and/or cleaned and/or irradiated proteins; (v) hydrolyzed unmodified proteins; (vi) hydrolyzed and chemically modified proteins, particularly hydrolyzed and hydrophobically and/or cationically and/or anionically modified proteins; (vii) hydrolyzed and physically modified proteins, particularly fractionated and/or cleaned and/or irradiated proteins; and (viii) mixtures thereof.

According to the invention, the term “unmodified proteins” should be understood to mean proteins that have been treated neither by means of chemical methods, such as hydrolysis or chemical modification, nor by means of physical methods, such as purification, separation, or irradiation.

Furthermore, according to the invention, the term “hydrolyzed proteins” or “protein hydrolysates” should be understood to mean proteins that are produced by chemical, particularly alkaline or acidic, hydrolysis, by enzymatic hydrolysis, and/or by a combination of both types of hydrolysis. All hydrolytically active enzymes, such as alkaline proteases, are suitable for enzymatic decomposition. Overviews of the production of protein hydrolysates have been presented, for example, by G. Schuster and A. Domsch in Seifen Öle Fette Wachse 108, (1982) 177 and Cosm. Toil. 99, (1984) 63, by H. W. Steisslinger in Parf. Kosm 72, (1991) 556, and by F. Aurich et al. in Tens. Surf. Det. 29, (1992) 389. Mixtures of individual amino acids that are obtained merely by mixing the pure substances of the amino acids and total hydrolysates that consist merely of individual amino acids do not fall under the term “hydrolyzed proteins” or “protein hydrolysates” in the context of the present invention.

Furthermore, in the context of the present invention, the term “chemically modified proteins” should be understood to mean proteins that are obtained by chemically reacting the reactive groups of the proteins, particularly the hydroxy, amine, imidazole, guanidino, and/or thiol groups of the side chains of the amino acids of the protein, with hydrophobic and/or cationic and/or anionic compounds.

In addition, in the sense of the present invention, the term “physically modified proteins” should be understood to mean proteins that have been modified by physical action, particularly by heat and/or light and/or fractionation.

In the context of this embodiment, it is especially preferred if the at least one protein is selected from the group of chemically modified, particularly hydrophobically modified, proteins. In this context, the hydrophobically modified protein has one or more C₄₋₃₀ hydrocarbon chains, wherein the C₄₋₃₀ hydrocarbon chains can be linear, cyclic, branched, unbranched, saturated, unsaturated, and aromatic and wherein the C₄₋₃₀ hydrocarbon chains are bonded to the protein residue by means of ether and/or ester and/or amine and/or amide bonds.

Furthermore, it is preferred in the context of this embodiment if the at least one protein is selected from the group of chemically modified, particularly cationically modified, proteins. The cationically modified protein therefore preferably includes one or more residues of the formula R¹—N⁺(CH₃)₂—CH₂—CH(OH)—CH₂—X—R, in which R¹ represents an alkyl group having 1 to 30 carbon atoms, an alkenyl group having 1 to 30 carbon atoms, a hydroxyalkyl group having 1 to 30 carbon atoms, particularly a methyl group, a C₁₀₋₁₈ alkyl, or a C₁₀₋₁₈ alkenyl group, X represents O, N, or S, and R represents the protein residue. The cationization of the proteins with the residues described above can be achieved by reacting the proteins, particularly the reactive groups of the amino acids of the proteins, with the corresponding halides of the formula above, wherein the residues described above can be bonded to the protein by means of ether and/or ester and/or amide and/or amine bonds. In the context of the present invention, the term “protein residue” should be understood to mean the backbone of the corresponding protein to which the cationic group is bonded by means of the previously mentioned bonds, which backbone is formed by the linking of amino acids.

According to another, preferred embodiment of the present invention, the at least one protein occurs in cutaneous appendages of mammals, particularly in the wool of sheep or goats.

In this context, it is especially preferred if the at least one protein is a keratin.

In the context of this embodiment, it is preferred if the at least one protein is a cationically functionalized and/or hydrolyzed keratin.

Furthermore, in the context of this embodiment, it is especially preferred if the at least one protein includes one or more residues of the formula R¹—N⁺(CH₃)₂—CH₂—CH(OH)—CH₂—X—R, in which R¹ represents a C₁₀₋₁₈ alkyl, or a C₁₀₋₁₈ alkenyl group, X represents O, N, or S, and R represents the possibly hydrolyzed protein residue.

With respect to the antiperspirant action, the skin compatibility, and the storage stability of the cosmetic agents according to the invention, it is especially preferred if the at least one protein is a keratin isolated from sheep's wool or goat's wool, wherein the keratin is hydrolyzed and/or wherein the keratin includes at least one lauryldimonium hydroxypropyl group. With no intention of being restricted to this theory, the use of these specific proteins results in a significantly increased effect on the sweat gland(s) due to pH-selective gel formation or due to disturbance of the charge equilibrium within the excretory ducts of the sweat glands. In this way, excellent antiperspirant action of the cosmetic agents according to the invention is ensured, which antiperspirant action is comparable to the antiperspirant action of aluminum-salt-containing or aluminum-zirconium-salt-containing cosmetic agents of the prior art. Furthermore, the use of these specific proteins does not lead to negative interaction with further ingredients in the antiperspirant cosmetic agent, and therefore high storage stability of the antiperspirant cosmetic agents according to the invention is ensured. In addition, the cosmetic agents according to the invention have high skin compatibility.

In this context, it has been found to be advantageous if the at least one protein includes lysine at 1.0 to 2.5 mol %, histidine at 0.5 to 0.8 mol %, and arginine at 7.0 to 8.0 mol %, with the respect to the total amount of substance of all amino acids of the protein. The amount-of-substance percentages of the previously mentioned amino acids can be determined, for example, chromatographically by using sulfonated polystyrene resins (S. Moore et al.; “Chromatography of amino acids on sulfonated polystyrene resin”; J. or Biological Chem., 1951, 192, pages 663 to 681).

Furthermore, it has proven to be advantageous in this context if the at least one protein has a calcium content of 100 to 600 mg, a potassium content of 700 to 1,400 mg, a phosphorus content of 40 to 150 mg, and a sulfur content of 1,100 to 5,700 mg, with respect to 1 kg of the at least one protein. The previously stated contents can be determined, for example, by means of atomic emission spectroscopy (ICP-OES) after microwave digestion with nitric acid (A. Oliveira et al.; “Evaluation of Metal Ions in Rice Samples: Extraction and Direct Determination by ICP-OES”; J. Braz. Chem. Soc., 2012, 23, pages 838 to 845).

According to another, especially preferred embodiment of the present invention, the at least one protein causes a change in the light absorption of 1.5 to 90%, preferably 2 to 80%, more preferably 2.5 to 70%, even more preferably 3 to 65%, particularly 3.5 to 60%. In particular, proteins from certain cutaneous appendages that cause the previously mentioned change in the light absorption lead to excellent antiperspirant action in the context of the present invention. The change in the light absorption can occur by a change in the light transmittance of the sample mixture, particularly as a result of opacification, and by the absorption of light by the sample mixture, particularly by the protein itself.

The changes in the light absorption in the event of a pH value change of at least 0.5, which form the basis of this invention, can be determined by measuring the light transmission of a light beam through the sample mixture. The measurements of the light transmission are performed in mV (resolution of 0.1 mV) at a wavelength of 574 nm (greenish yellow) in an open sample vessel at 23° C. and 1,013 mbar by using a Metrohm Optrode 6.1115.000. The pH value change in the pH range of 4.0 to 8.0 is achieved by slowly and continuously adding a carbonate or hydrogencarbonate solution, preferably a 1 wt % sodium hydrogencarbonate solution, to the sample mixture while measuring the pH value with a pH electrode and while stirring at a speed of 750 to 850 rpm. The change in the light absorption that is caused by the at least one protein is calculated in accordance with the formula ΔL=[(Lᵢ|/|L₀|]*100. In this formula, Lᵢ represents the light transmission after a change in the pH value by at least 0.5 in the pH range of 4.0 to 8.0, preferably pH 4.5 and 7.5, particularly pH 5.0 and 7.0. In this formula, L₀ represents the difference of the light transmission at pH 4.0 and at pH 8.0, preferably at pH 4.5 and at pH 7.5, particularly at pH 5.0 and at pH 7.0, for example light transmission at pH 8.0 minus light transmission at pH 4.0. The at least one specific protein in the antiperspirant cosmetic agents according to the invention causes a change in the light absorption of 1 to 100%, said change being determined in accordance with the method above. However, the present invention is not restricted to antiperspirant cosmetic preparations that include at least one specific protein that causes a change in the light absorption of 1 to 100%, said change being determined in accordance with the method above. The present invention also comprises antiperspirant cosmetic compositions that include at least one specific protein that causes a change in the light absorption of 1 to 100% in accordance with other methods.

In the context of the present invention, it is preferred if the concentration of the at least one protein in the sample mixture used to determine the change in the light absorption is 0.005 to 10 wt %, preferably 0.05 to 5 wt %, more preferably 0.07 to 3 wt %, particularly 0.09 to 2 wt %, with respect to the total weight of the sample mixture used to determine the change in the light absorption.

According to the invention, the at least one protein preferably causes a change in the light absorption in the event of a pH value change of at least 0.5 and at most 3.5, more preferably at least 0.5 and at most 2.5, particularly at least 0.5 and at most 1.5. In particular, the change in the pH value can be achieved by adding acids or bases, preferably bases in the form of carbonates or hydrogencarbonates, in the appropriate amount.

According to another, preferred embodiment of the present invention, the antiperspirant cosmetic agent has a pH value of pH 2 to pH 10. Within this range, a stable formulation of the cosmetic agents according to the invention is possible without the occurrence of undesired interactions between the ingredients of the antiperspirant cosmetic agents according to the invention. According to the invention, the desired pH value can be set by using acids and bases that are known to a person skilled in the art and are common in antiperspirant cosmetic agents.

According to the invention, it is also preferred if the antiperspirant cosmetic agent additionally includes at least one preservative agent. Preservative agents preferred according to the invention are formaldehyde releaser, iodopropynyl butylcarbamate, parabens, phenoxyethanol, ethanol, benzoic acid and salts thereof, dibromodicyanobutane, 2-bromo-2-nitropropane-1,3-diol, imidazolidinyl urea, 5-chloro-2-methyl-4-isothiazolin-3-one, 2-chloroacetamide, benzalkonium chloride, benzyl alcohol, salicylic acid, and salicylates. Other preservative agents that are usable in the context of the present invention are the substances listed in Annex 6 of the Cosmetics Regulation and cosmetic raw substances having preservative properties or raw substances that support or intensify the preservative action of the aforementioned preservative agents. The preservative agents are preferably included in a total amount of 0.01 to 10 wt %, preferably 0.1 to 7 wt %, more preferably 0.2 to 5 wt %, particularly 0.3 to 2.0 wt %, with respect to the total weight of the antiperspirant cosmetic agent.

In the context of the present invention, it is preferred if the antiperspirant cosmetic agent exists in the form of a water-in-oil emulsion. In particular, the water-in-oil emulsion can be a sprayable water-in-oil emulsion, which can be sprayed by means of a propellant. In this context, it is preferred if the antiperspirant cosmetic agent existing in the form of a water-in-oil emulsion includes the at least one protein in a total amount of 0.1 to 70 wt %, preferably 0.5 to 60 wt %, more preferably 1.0 to 50 wt %, even more preferably 1.5 to 40 wt %, even more preferably 2.0 to 30 wt %, particularly 2.0 to 20 wt %, with respect to the total weight of the antiperspirant cosmetic agent.

However, it can be equally preferred according to the invention if the antiperspirant cosmetic agent exists as an oil-in-water emulsion. In this case, the cosmetic agent according to the invention is preferably sprayed as a propellant-free pump spray or squeeze spray or applied as a roll-on. In this context, it is preferred if the antiperspirant cosmetic agent existing in the form of an oil-in-water emulsion includes the at least one protein in a total amount of 0.1 to 70 wt %, preferably 0.5 to 60 wt %, more preferably 1.0 to 50 wt %, even more preferably 1.5 to 40 wt %, even more preferably 2.0 to 30 wt %, particularly 2.0 to 20 wt %, with respect to the total weight of the antiperspirant cosmetic agent.

According to another, preferred embodiment of the present invention, the cosmetic agents according to the invention can include only a small content of free water or no free water. In the sense of the present invention, the term “free water” is understood to mean water that is different from water of crystallization, hydration water, or similarly molecularly bound water of the constituents that are used. The antiperspirant cosmetic agent preferably includes free water in a total amount of less than 10 wt %, preferably less than 8 wt %, more preferably less than 5 wt %, even more preferably less than 3 wt %, even more preferably less than 1 wt %, particularly 0 wt %, with respect to the total weight of the antiperspirant cosmetic agent.

However, in the context of a further embodiment, it is also preferred according to the invention if the antiperspirant cosmetic agent exists as an aqueous, aqueous-alcoholic, or aqueous-glycolic solution. According to the invention, because the cosmetic agents according to the invention include no antiperspirant aluminum and/or zirconium halides and/or hydroxyhalides, which have reduced antiperspirant action as a result of the addition of protic solvents, protic solvents such as aqueous solutions can be used to formulate the antiperspirant cosmetic agents according to the invention without the occurrence of a significant reduction in the antiperspirant action. Therefore, the addition of the at least one specific protein ensures effective influencing of the sweat gland(s) and thus excellent antiperspirant action even if protic solvents are used.

In the context of this embodiment of the present invention, it was surprisingly found that the effect on the sweat gland(s) by the at least one specific protein can be significantly increased if the antiperspirant cosmetic agents according to the invention include free water in an amount of 5 to 99 wt %, with respect to the total weight of the antiperspirant cosmetic agent. Therefore, in an especially preferred embodiment of the present invention, the antiperspirant cosmetic agent includes free water in a total amount of 5 to 96 wt %, preferably 15 to 80 wt %, more preferably 30 to 70 wt %, particularly 40 to 60 wt %, with respect to the total weight of the antiperspirant cosmetic agent.

Furthermore, in the context of this embodiment, it is preferred if the antiperspirant cosmetic agent includes ethanol in a total amount of 1 to 99 wt %, preferably 5 to 70 wt %, preferably 7 to 50 wt %, particularly 10 to 30 wt %, with respect to the total weight of the antiperspirant cosmetic agent. As previously stated, because of the use of the at least one specific protein, even high amounts of protic solvents such as ethanol can be used without the antiperspirant action of the antiperspirant cosmetic agent according to the invention being negatively affected.

The antiperspirant cosmetic agent according to the invention can be applied by means of various methods. According to a preferred embodiment, the antiperspirant cosmetic agent is formulated as a spray application. The spray application is accomplished by means of a spraying device, which includes a filling of the antiperspirant cosmetic agent according to the invention, which is liquid, viscously flowable, in the form of a suspension, or in the form of a powder, in a container. The filling can be under the pressure of a propellant (compressed-gas cans, compressed-gas packages, aerosol packages), or a mechanically operated pump atomizer without propellant gas (pump sprays/squeeze bottle) can be used. The antiperspirant cosmetic agent can be atomized physically, mechanically, or electromechanically, for example by means of piezoelectric effects or electric pumps. Containers and removal devices that are usable in the context of this embodiment are described, for example, in laid-open application DE 102012222692 A1.

Furthermore, the antiperspirant cosmetic agent can preferably be formulated as a stick, soft solid, cream, gel, roll-on, or loose or compact powder. The formulation of the antiperspirant cosmetic agents according to the invention in a certain product form, such as an antiperspirant roll-on, an antiperspirant stick, or an antiperspirant gel, is preferably based on the requirements of the intended use. Therefore, depending on the intended use, the antiperspirant cosmetic agents according to the invention can exist in solid, semisolid, liquid, disperse, emulsified, suspended, gel, multi-phase, or powdery form. In the sense of the present invention, all types of solid dispersions in liquids also fall under the term “liquid.” Furthermore, in the sense of the present invention, agents that have at least two different phases having a phase separation and in the case of which the phases can be arranged horizontal, i.e., one over the other, or vertical, i.e., one next to the other, are understood by multi-phase antiperspirant cosmetic agents according to the invention. The application can be performed, for example, by means of a roller-ball applicator or by means of a solid stick.

In the context of the present invention, it can also be preferred if the antiperspirant cosmetic agent is included on or in a disposable substrate, selected from the group of wipes, pads, and puffs. Especially preferred are wet wipes, i.e., preferably individually packaged wet wipes prefabricated for the user, which are well known, for example, from the field of glass cleaning or from the field of wet toilet wipes. Such wet wipes, which can advantageously also include preservative substances, are impregnated or loaded with an antiperspirant cosmetic agent according to the invention and are preferably packaged individually. Preferred substrate materials are selected from porous flat wipes. These wipes include wipes composed of woven and nonwoven synthetic and natural fibers, felt, paper, or foam, such as hydrophilic polyurethane foam. Deodorizing or antiperspirant substrates preferred according to the invention can be obtained by soaking or impregnation or by applying a cosmetic agent according to the invention to a substrate in melted form.

According to the invention, it is preferred that the antiperspirant cosmetic agent includes at least one further auxiliary substance, selected from the group of (i) emulsifiers and/or surfactants; (ii) thickeners; (iii) chelating agents; (iv) deodorant active substances; (v) mono- and/or polyhydric alcohols and/or polyethylene glycols; (vi) skin-cooling active substances; (vii) pH adjusters; (viii) skin care active substances, such as moisturizers, skin-soothing active substances, skin-lightening substances, skin-smoothing substances; and (ix) mixtures thereof.

Suitable emulsifiers and surfactants preferred according to the invention are selected from anionic, cationic, non-ionic, amphoteric, particularly ampholytic and zwitterionic emulsifiers and surfactants. Surfactants are amphiphilic (bifunctional) compounds that consist of at least one hydrophobic molecule part and at least one hydrophilic molecule part. The hydrophobic residue is preferably a hydrocarbon chain having 8 to 28 carbon atoms, which hydrocarbon chain can be saturated or unsaturated, linear or branched. This C₈-C₂₈ alkyl chain is especially preferably linear. Emulsifiers and surfactants that are usable with preference in the context of the present invention are disclosed, for example, in laid-open applications DE 102012222692 A1, DE 102010063250 A1, and DE 102010055816 A1.

To thicken the antiperspirant cosmetic agents according to the invention, preferably substances selected from the following are used: cellulose ethers, xanthan gum, sclerotium gum, succinoglucans, polygalactomannans, pectins, agar, carrageenan, tragacanth, gum arabic, gum karaya, tara gum, gellan gum, gelatin, propylene glycol alginate, alginic acids and salts thereof, polyvinylpyrrolidones, polyvinyl alcohols, polacrylamides, starches that are physically modified (e.g., by means of pre-gelatinization) and/or chemically modified, acrylic acid/acrylate copolymers, acrylic acid/acrylamide copolymers, acrylic acid/vinylpyrrolidone copolymers, acrylic acid/vinylformamide copolymers, and polyacrylates. Furthermore, especially preferred thickeners are selected from carbomers. Carbomers are thickening cross-linked polymers of acrylic acid, methacrylic acid, and salts thereof. The cross-linking can be accomplished by means of polyfunctional compounds such as polyalkylene ethers of polysaccharides or of polyalcohols, such as sucrose allyl ethers, pentaerythritol allyl ethers, propylene allyl ethers. Homopolymers of acrylic acid or salts thereof that are cross-linked by means of a pentaerythritol allyl ether, a sucrose allyl ether, or a propylene allyl ether are preferred in the context of the present invention. A copolymer of C₁₀₋₃₀ alkyl acrylate, acrylic acid, methacrylic acid, and esters thereof that is cross-linked by means of a sucrose allyl ether or a pentaerythritol allyl ether is a thickener that is usable in the context of the present invention. The products available under the trade name Carbopol® (BF Goodrich, Ohio, USA), such as Carbopol 934, Carbopol 940, Carbopol 941, Carbopol 971, Carbopol 974, Carbopol EZ2, Carbopol ETD 2001, Carbopol ETD 2020, Carbopol ETD 2050, Carbopol ultrez 10, Carbopol ultrez 20, or Carbopol ultrez 21 are thickeners based on carbomers.

Furthermore, lipophilic thickeners can be used to thicken the antiperspirant cosmetic agents according to the invention. Lipophilic thickeners preferred according to the invention are selected from hydrophobed clay minerals, bentonites, pyrogenic silicic acids, and derivatives thereof.

To further support the effect of the at least one specific protein on the sweat gland(s), it can be advantageous to add at least one chelating agent to the antiperspirant cosmetic agents according to the invention in a total amount of 0.01 to 3.0 wt %, preferably 0.02 to 1.0 wt %, particularly 0.05 to 0.1 wt %, with respect to the total weight of the antiperspirant agent according to the invention. In the context of the present invention, preferred chelating agents are selected from the group of β-alanine diacetic acid, cyclodextrin, diethylenetriamine penta(methylene phosphonic acid), sodium, potassium, calcium disodium, ammonium, and triethanolamine salts of ethylenediamine tetraacetic acid (EDTA), etidronic acid, hydroxyethyl ethylenediamine tetraacetic acid (HEDTA) and sodium salts thereof, sodium salts of nitrilotriacetic acid (NTA), diethylenetriamine pentaacetic acid, phytic acid, hydroxypropyl cyclodextrin, methyl cyclodextrin, pentasodium amino trimethylene phosphonate, pentasodium ethylenediamine tetramethylene phosphonate, pentasodium diethylenetriaminepentaacetate, pentasodium triphosphate, potassium EDTMP, sodium EDTMP, sodium dihydroxyethylglycinate, sodium phytate, sodium polydimethylglycinophenol sulfonate, tetrahydroxyethyl ethylenediamine, tetrahydroxypropyl ethylenediamine, tetrapotassium etidronate, tetrasodium etidronate, tetrasodium iminodisuccinate, trisodium ethylenediamine disuccinate, tetrasodium N,N-bis(carboxymethyl)-glutamate, tetrasodium DL-alanine-N,N-diacetate, and desferrioxamine.

The deodorizing action of the antiperspirant cosmetic agents according to the invention can be increased further if at least one deodorant active substance having antibacterial and/or bacteriostatic and/or enzyme-inhibiting and/or odor-neutralizing and/or odor-absorbing action is included in a total amount of 0.0001 to 40 wt %, preferably 0.2 to 20 wt %, more preferably 1 to 15 wt %, particularly 1.5 to 5 wt %, with respect to the total weight of the antiperspirant cosmetic agent according to the invention. If ethanol is used in the agents according to the invention, the ethanol is not considered to be a deodorant active substance in the context of the present invention, but rather a constituent of the carrier. Deodorant active substances preferred according to the invention are disclosed, for example, in laid-open application DE 102010063250 A1.

Preferred antiperspirant cosmetic agents according to the invention also include at least one water-soluble polyhydric C₂₋₉ alkanol having 2 to 6 hydroxyl groups and/or at least one water-soluble polyethylene glycol having 3 to 50 ethylene oxide units and mixtures thereof. The aforementioned deodorant active substances in the form of 1,2-alkanediols do not fall thereunder. Preferred alkanols and water-soluble polyethylene glycols are described, for example, in laid-open application DE 102010063250 A1.

According to another embodiment of the present invention, the antiperspirant cosmetic agents also include at least one skin-cooling active substance. Skin-cooling active substances suitable according to the invention are, for example, menthol, isopulegol, and menthol derivatives, e.g., menthyl lactate, menthyl glycolate, menthyl ethyl oxamate, menthyl pyrrolidone carboxylic acid, menthyl methyl ether, menthoxypropanediol, menthone glycerin acetal (9-methyl-6-(1-methylethyl)-1,4-dioxaspiro[4.5]decane-2-methanol), monomenthyl succinate, 2-hydroxymethyl-3,5,5-trimethylcyclohexanol, and 5-methyl-2-(1-methylethyl)cyclohexyl-N-ethyloxamate. Menthol, isopulegol, menthyl lactate, menthoxypropanediol, menthyl pyrrolidone carboxylic acid, and 5-methyl-2-(1-methylethyl)cyclohexyl-N-ethyloxamate and mixtures of these substances, particularly mixtures of menthol and menthyl lactate, menthol, menthol glycolate, and menthyl lactate, menthol and menthoxypropanediol, or menthol and isopulegol, are preferred as skin-cooling active substances.

According to the invention, preferably acids and/or alkalizing agents and/or buffers are used as pH adjusters. According to the invention, preferably inorganic acids (such as hydrochloric acid, sulfuric acid, or phosphoric acid) or organic acids (such as citric acid, tartaric acid, or malic acid) are used as acids. The alkalizing agents that are usable according to the invention are preferably selected from the group consisting of ammonia, basic amino acids, alkali hydroxides, carbonates and hydrogencarbonates, alkanolamines, such as amino-2-methyl-1-propanol, monoethanolamine, triethanolamine, diethanoamine, and triisopropanolamine, alkali metal metasilicates, urea, morpholine, N-methylglucamine, imidazole, alkali phosphates, and alkali hydrogenphosphates. Lithium, sodium, potassium, particularly sodium or potassium, is used as an alkali metal ion. In particular, carbonic acid/bicarbonate buffer, carbonic acid/silicate buffer, acetic acid/acetate buffer, phosphate buffer, ammonia buffer, citric acid or citrate buffer, buffer based on tris(hydroxymethyl)aminomethane, buffer based on 4-(2-hydroxyethyl)-1-piperazineethanesulfonic acid, buffer based on 4-(2-hydroxyethyl)piperazine-1-propanesulfonic acid, buffer based on 2-(N-morpholino)ethanesulfonic acid, and barbital/acetate buffer are suitable as buffer systems in the context of the present invention. The appropriate buffer system is selected on the basis of the desired pH value of the antiperspirant cosmetic agents according to the invention.

In a preferred embodiment, the antiperspirant cosmetic agents according to the invention are characterized in that said agents include—with respect to the total weight of the antiperspirant cosmetic agent according to the invention—
at least one protein in a total amount of 0.5 to 60 wt %, preferably 1.0 to 50 wt %, more preferably 1.5 to 40 wt %, even more preferably 2.0 to 30 wt %, particularly 2.0 to 20 wt %,
12 to 98 wt %, preferably 25 to 55 wt %, more preferably 30 to 50 wt %, particularly 35 to 45 wt %, of water,
at least one emulsifier and/or one surfactant,
at least one pH adjuster,
at least one preservative agent, and
at least one substance selected from the group of cosmetic oils that are liquid at 20° C. and 1,013 hPa, odorous substances, and waxes,
   wherein the at least one protein occurs in cutaneous appendages of mammals, birds, fish, insects, or crustaceans and wherein the at least one protein causes a change in the light absorption of 1 to 100% in the event of a pH value change of at least 0.5 in a pH range of pH 4.0 to pH 8.0 at a temperature of 20° C. to 40° C. and a concentration of the protein of 0.001 to 10 wt %, with respect to the total weight of the sample mixture used to determine the change in the light absorption.

In another preferred embodiment, the antiperspirant cosmetic agents according to the invention are characterized in that said agents include—with respect to the total weight of the antiperspirant cosmetic agent according to the invention—
at least one protein in a total amount of 0.5 to 60 wt %, preferably 1.0 to 50 wt %, more preferably 1.5 to 40 wt %, even more preferably 2.0 to 30 wt %, particularly 2.0 to 20 wt %,
12 to 98 wt %, preferably 25 to 55 wt %, more preferably 30 to 50 wt %, particularly 35 to 45 wt %, of water,
at least one emulsifier and/or one surfactant,
at least one pH adjuster,
at least one preservative agent,
0.01 to 2 wt %, preferably 0.1 to 1 wt %, more preferably 0.2 to 0.7 wt %, particularly 0.3 to 0.5 wt %, of a thickener, and
at least one substance selected from the group of cosmetic oils that are liquid at 20° C. and 1,013 hPa, odorous substances, and waxes,
   wherein the at least one protein occurs in cutaneous appendages of mammals, birds, fish, insects, or crustaceans and wherein the at least one protein causes a change in the light absorption of 1 to 100% in the event of a pH value change of at least 0.5 in a pH range of pH 4.0 to pH 8.0 at a temperature of 20° C. to 40° C. and a concentration of the protein of 0.001 to 10 wt %, with respect to the total weight of the sample mixture used to determine the change in the light absorption.

In a preferred embodiment, the antiperspirant cosmetic agents according to the invention are characterized in that said agents include—with respect to the total weight of the antiperspirant cosmetic agent according to the invention—
at least one protein in a total amount of 0.5 to 60 wt %, preferably 1.0 to 50 wt %, more preferably 1.5 to 40 wt %, even more preferably 2.0 to 30 wt %, particularly 2.0 to 20 wt %,
12 to 98 wt %, preferably 25 to 55 wt %, more preferably 30 to 50 wt %, particularly 35 to 45 wt %, of water,
at least one propellant in a total amount of 1 to 98 wt %, preferably 20 to 90 wt %, more preferably 30 to 85 wt %, particularly 40 to 75 wt %,
at least one emulsifier and/or one surfactant,
at least one pH adjuster,
at least one preservative agent, and
at least one substance selected from the group of cosmetic oils that are liquid at 20° C. and 1,013 hPa, odorous substances, and waxes,
   wherein the at least one protein occurs in cutaneous appendages of mammals, birds, fish, insects, or crustaceans and wherein the at least one protein causes a change in the light absorption of 1 to 100% in the event of a pH value change of at least 0.5 in a pH range of pH 4.0 to pH 8.0 at a temperature of 20° C. to 40° C. and a concentration of the protein of 0.001 to 10 wt %, with respect to the total weight of the sample mixture used to determine the change in the light absorption.

In another preferred embodiment, the antiperspirant cosmetic agents according to the invention are characterized in that said agents include—with respect to the total weight of the antiperspirant cosmetic agent according to the invention—
at least one protein in a total amount of 0.5 to 60 wt %, preferably 1.0 to 50 wt %, more preferably 1.5 to 40 wt %, even more preferably 2.0 to 30 wt %, particularly 2.0 to 20 wt %,
12 to 98 wt %, preferably 25 to 55 wt %, more preferably 30 to 50 wt %, particularly 35 to 45 wt %, of water,
at least one propellant in a total amount of 1 to 98 wt %, preferably 20 to 90 wt %, more preferably 30 to 85 wt %, particularly 40 to 75 wt %,
at least one emulsifier and/or one surfactant,
at least one pH adjuster,
at least one preservative agent,
0.01 to 2 wt %, preferably 0.1 to 1 wt %, more preferably 0.2 to 0.7 wt %, particularly 0.3 to 0.5 wt %, of a thickener, and
at least one substance selected from the group of cosmetic oils that are liquid at 20° C. and 1,013 hPa, odorous substances, and waxes,
   wherein the at least one protein occurs in cutaneous appendages of mammals, birds, fish, insects, or crustaceans and wherein the at least one protein causes a change in the light absorption of 1 to 100% in the event of a pH value change of at least 0.5 in a pH range of pH 4.0 to pH 8.0 at a temperature of 20° C. to 40° C. and a concentration of the protein of 0.001 to 10 wt %, with respect to the total weight of the sample mixture used to determine the change in the light absorption.

In the context of the present invention, it can also be provided that the cosmetic agent according to the invention is formulated as a two-component agent. For this purpose, the individual components are preferably stored in separate containers and are applied to the skin one after the other in any order or simultaneously. Separation into multi-component systems is preferred particularly if incompatibility of the ingredients is expected or feared.

Therefore, another subject of the present invention is a packaging unit (kit of parts), comprising—formulated separate from each other—
a) at least one first container (C1), containing a cosmetic agent (M1) comprising at least one antiperspirant active substance, and
b) at least one second container (C2), containing a cosmetic agent (M2) comprising at least one protein, wherein the at least one protein occurs in cutaneous appendages of mammals, birds, fish, insects, or crustaceans, wherein the at least one protein causes a change in the light absorption of 1 to 100% in the event of a pH value change of at least 0.5 in a pH range of pH 4.0 to pH 8.0 at a temperature of 20° C. to 40° C. and a concentration of the protein of 0.001 to 10 wt %, with respect to the total weight of the sample mixture used to determine the change in the light absorption, and wherein the cosmetic agent (M2) includes no aluminum and/or zirconium halides and/or hydroxyhalides.

According to the invention, the term “antiperspirant active substance” is understood to mean active substances that reduce the perspiration of the sweat glands of the body. However, the proteins from cutaneous appendages of mammals, birds, fish, insects, or crustaceans that are included in the cosmetic agent (M2) and that cause a change in the light absorption of 1 to 100% under the conditions described above do not fall thereunder.

The statements made with respect to the cosmetic agents according to the invention apply, mutatis mutandis, to the cosmetic agent (M2) in the container (C2).

Another subject of the present invention is the use of a protein to at least partially influence the sweat gland(s), wherein the at least one protein occurs in cutaneous appendages of mammals, birds, fish, insects, or crustaceans and wherein the at least one protein causes a change in the light absorption of 1 to 100% in the event of a pH value change of at least 0.5 in a pH range of pH 4.0 to pH 8.0 at a temperature of 20° C. to 40° C. and a concentration of the protein of 0.001 to 10 wt %, with respect to the total weight of the sample mixture used to determine the change in the light absorption.

According to the invention, “to influence the sweat gland(s)” should be understood to mean to influence the sweat gland(s) in such a way that the secretion of sweat from the excretory duct is avoided or reduced. With no intention of being restricted to one theory, this can be accomplished, for example, by the formation of a gel and/or precipitate of the at least one specific protein in the excretory duct of the sweat gland or the excretory ducts of the sweat glands. Furthermore, the use of the at least one specific protein can, however, also lead to disturbance of the charge equilibrium within the excretory ducts of the sweat glands. The statements made with respect to the cosmetic antiperspirant agents according to the invention apply, mutatis mutandis, to the use according to the invention.

Furthermore, another subject of the present invention is the use of a combination, including
a) at least one substance selected from the group of cosmetic oils that are liquid at 20° C. and 1,013 hPa, odorous substances, and waxes,
b) propellant in a total amount of 0 to 99 wt %, with respect to the total weight of the antiperspirant cosmetic agent, and
c) at least one protein in a total amount of 0.1 to 70 wt %, with respect to the total weight of the antiperspirant cosmetic agent, wherein the at least one protein occurs in cutaneous appendages of mammals, birds, fish, insects, or crustaceans, wherein the at least one protein causes a change in the light absorption of 1 to 100% in the event of a pH value change of at least 0.5 in a pH range of pH 4.0 to pH 8.0 at a temperature of 20° C. to 40° C. and a concentration of the protein of 0.001 to 10 wt %, with respect to the total weight of the sample mixture used to determine the change in the light absorption, and
   wherein the combination includes no aluminum and/or zirconium halides and/or hydroxyhalides,
   to reduce and/or prevent sweat, particularly axillary sweat or sweat of other body regions.

In the sense of the present invention, the term “combination” comprises a mixture of the ingredients a), b), and c) that are specified above. The statements made with respect to the antiperspirant cosmetic agents according to the invention and to the use according to the invention apply, mutatis mutandis, to the use according to the use of the aforementioned combination.

In addition, another subject of the present invention is an antiperspirant cosmetic agent, including
a) at least one substance selected from the group of cosmetic oils that are liquid at 20° C. and 1,013 hPa, odorous substances, and waxes,
b) propellant in a total amount of 0 to 99 wt %, with respect to the total weight of the antiperspirant cosmetic agent, and
c) at least one keratin isolated from sheep's wool or goat's wool in a total amount of 0.1 to 70 wt %, with respect to the total weight of the antiperspirant cosmetic agent, wherein the keratin is hydrolyzed and/or wherein the keratin includes at least one lauryldimonium hydroxypropyl group,
   wherein the antiperspirant cosmetic agent includes no aluminum and/or zirconium halides and/or hydroxyhalides.

In the context of this subject, it is particularly preferred if the at least one protein includes lysine at 1.0 to 2.5 mol %, histidine at 0.5 to 0.8 mol %, and arginine at 7.0 to 8.0 mol %, with the respect to the total amount of substance of all amino acids of the protein. The content of the mentioned amino acids can be determined as previously stated.

Furthermore, it is especially preferred in the context of this subject if the at least one protein has a calcium content of 100 to 600 mg, a potassium content of 700 to 1,400 mg, a phosphorus content of 40 to 150 mg, and a sulfur content of 1,100 to 5,700 mg, with respect to 1 kg of the at least one protein. The stated contents can be determined, for example, by means of atomic emission spectroscopy (ICP-OES) as previously stated.

The statements made with respect to the antiperspirant cosmetic agents according to the invention, the use according to the invention, and the method according to the invention apply, mutatis mutandis, to other, preferred embodiments of this subject.

Finally, another subject of the present invention is a non-therapeutic cosmetic method for preventing and/or reducing the perspiration of the body, wherein an antiperspirant cosmetic agent according to the invention is applied to a skin surface, particularly to the skin of the axillae, and remains on the skin surface for at least 1 hour, preferably for at least 2 hours, preferably for at least 4 hours, particularly for at least 6 hours.

In the context of the method according to the invention, it can also be provided, however, that first a cosmetic agent including at least one antiperspirant aluminum and/or zirconium halide and/or hydroxyhalide is applied and then the cosmetic agent according to the invention is applied. But it is also possible that first the cosmetic agent according to the invention is applied and then a cosmetic agent including at least one antiperspirant aluminum and/or zirconium halide and/or hydroxyhalide is used. Furthermore, the antiperspirant cosmetic agent according to the invention and the cosmetic agent including at least one antiperspirant aluminum and/or zirconium halide and/or hydroxyhalide can also be applied to the skin at the same time. The time span between the application of the two agents is 0 seconds to 24 hours. Furthermore, it is preferred if the agents remain on the skin of the axillae for at least 1 hour, preferably for at least 2 hours, more preferably for at least 4 hours, particularly for at least 6 hours.

The statements made with respect to the antiperspirant cosmetic agents according to the invention and to the use according to the invention apply, mutatis mutandis, to the method according to the invention.

The following examples illustrate the present invention without restricting the present invention to said examples:

### Examples

### 1. Change in the Light Absorption

The change in the light absorption caused by a hydrolyzed keratin having at least one lauryldimonium hydroxypropyl group and an average molecular weight M_{w} of approximately 1,000 Da in a pH range of 4.5 to 7.5 in the event of a pH value change of 1.0 is determined as follows:

TABLE 1
Sample solution (specifications in wt %)

| | E-I* |
|---|---|
| Hydrolyzed keratin having at least one | 4 |
| lauryldimonium hydroxypropyl group | |
| HCl | ad pH |
| Water | ad 100 |

*according to the invention
^{a)}Croquat WKP PE-LQ (WD); (INCI: Cocodimonium Hydroxypropyl Hydrolyzed Keratin; 30 wt % solution in water; M_{w} approximately 1,000 Da; Croda)

For the determination of the change in the light absorption, a Methrom Titrando 905 from the company Methrom (USA) was used, which is equipped with a Methrom Optrode 6.1115.000 and a pH electrode from Methrom. The Methrom Titrando 905 is controlled by means of the Tiamo software from Methrom. First, 30 mL of a sample solution according to table 1, which has a pH value of 3.0, was provided in the open sample vessel of the Methrom Titrando 905. Then, at 23° C. and 1,013 mbar and while stirring (stirring speed 8 of the Titrando 905, corresponds to 750 to 850 rpm), a 1 wt % sodium hydrogencarbonate solution was added continuously until a pH value of 7.5 was reached. During the addition of the 1 wt % sodium hydrogencarbonate solution, the light transmission of a light beam through said sample solution was measured in mV (resolution of 0.1 mV) at a wavelength of 574 nm (greenish yellow) by using a Methrom Optrode 6.1115.000. Each measurement is performed twice and the mean is calculated therefrom.

The change in the light absorption that was caused by the aforementioned peptides was determined in accordance with the formula ΔL=[(|Lᵢ|/|L₀|]*100. In this formula, Lᵢ represents the light transmission after a change in the pH value by at least 0.5 in the pH range of 4.0 to 8.0, preferably pH 4.5 and 7.5, particularly pH 5.0 and 7.0. In this formula, L₀ represents the difference of the light transmission at pH 4.0 and at pH 8.0, preferably at pH 4.5 and at pH 7.5, particularly at pH 5.0 and at pH 7.0.

In the event of a pH value change of 1.0 between pH 5.0 and pH 6.0 (light absorption at pH 6.0 forms the value Lᵢ) in a pH value range of 4.5 to 7.5 (difference of light absorption at pH 7.5 minus light absorption at pH 4.5 forms the value L₀), said protein caused a change in the light absorption ΔL of approximately 50%.

### 2. In Vivo Test for Antiperspirant Action

To determine the antiperspirant action, an antiperspirant study was performed on the backs of 16 female test subjects. The following antiperspirant agents were used for this purpose:

| Antiperspirant agent | No. |
|---|---|
| Aqueous solution having 10% ACH, pH 4 | V-I |
| Aqueous solution having 5%* protein^{a)}, pH 2-4 | E-II** |

*active substance
**according to the invention
^{a)}Croquat WKP PE-LQ (WD); (INCI: Cocodimonium Hydroxypropyl Hydrolyzed Keratin; 30 wt % solution in water; M_{w} approximately 1,000 Da; Croda)

On the back of each of 16 female test subjects, 40 μL of the antiperspirant agent V-I and 75 μL of the cosmetic agent E-II according to the invention were applied on one side next to the spine. After 5 minutes, the treated locations were covered with occlusive non-adsorbent film. After 2 hours, these non-adsorbent pads were removed. The compositions were applied to the backs of the female test subjects in the previously described manner on four consecutive days. 24 h after the last application of the composition, absorbent pads were applied to the backs of the female test subjects in the locations where the compositions had previously been applied. Furthermore, pads serving as a control were also applied on the other side of the spine at the same height. After the female test subjects had sweated in a sauna for approximately 15 minutes at 80° C., the amount of sweat absorbed by the pads was determined gravimetrically, wherein each composition was compared with the corresponding untreated location on the back. The sweat reduction was determined from the gravimetric determination of the sweat amount, wherein all determined values were statistically significant.

The sweat reduction of each composition in comparison with an untreated skin location is presented in the following table:

| No. | Sweat reduction |
|---|---|
| V-I | 50% |
| E-II | 17% |

The use of the specific protein leads to a significant decrease in the sweat reduction and to satisfactory antiperspirant action.

### 3. Formulations

The protein used in the following examples is preferably a hydrolyzed keratin having at least one lauryldimonium hydroxypropyl group and an average molecular weight M_{w} of approximately 1,000 Da:

Antiperspirant cosmetic agents according to the invention having a pH of 2.5 to 10.0 (amount specifications in wt %)

| | 1 | 2 | 3 | 4 | 5 | 6 | 7 |
|---|---|---|---|---|---|---|---|
| Isopropyl myristate | 0.50 | 0.10 | 0.50 | 1.0 | 2.0 | 3.0 | 5.0 |
| Protein | 0.50 | 2.0 | 3.0 | 5.0 | 7.0 | 10 | 20 |
| Eumulgin B3 ^{b)} | 3.0 | 3.0 | 3.0 | 4.0 | 4.0 | 4.0 | 5.0 |
| Perfume | 0.10 | 0.20 | 0.30 | 0.30 | 0.50 | 0.8 | 1.0 |
| Preservative agent | 0.50 | 0.50 | 0.50 | 0.80 | 0.80 | 1.5 | 2.0 |
| pH adjuster | ad pH | ad pH | ad pH | ad pH | ad pH | ad pH | ad pH |
| Water | ad 100 | ad 100 | ad 100 | ad 100 | ad 100 | ad 100 | ad 100 |

^{b)} Eumulgin B3 (INCI: Ceteareth-30; BASF)

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. An antiperspirant cosmetic agent, comprising
a) at least one substance selected from the group consisting of cosmetic oils that are liquid at 20° C. and 1,013 hPa, odorous substances, and waxes,
b) propellant in a total amount of 0 to 99 wt %, with respect to the total weight of the antiperspirant cosmetic agent, and
c) at least one protein in a total amount of 0.1 to 70 wt %, with respect to the total weight of the antiperspirant cosmetic agent, wherein the at least one protein occurs in cutaneous appendages of mammals, birds, fish, insects, or crustaceans and wherein the at least one protein causes a change in the light absorption of 1 to 100% in the event of a pH value change of at least 0.5 in a pH range of pH 4.0 to pH 8.0, at a temperature of 20° C. to 40° C. and a concentration of the protein of 0.001 to 10 wt %, with respect to the total weight of the sample mixture used to determine the change in the light absorption,
wherein the antiperspirant cosmetic agent includes no aluminum and/or zirconium halides and/or hydroxyhalides.

2. The antiperspirant cosmetic agent according to claim 1, wherein the at least one protein is included in a total amount of 0.5 to 60 wt % based on the total weight of the antiperspirant cosmetic agent.

3. The antiperspirant cosmetic agent according to claim 1, wherein the at least one protein is included in a total amount of 1.5 to 50 wt based on the total weight of the antiperspirant cosmetic agent.

4. The antiperspirant cosmetic agent according to claim 1, wherein the at least one protein is included in a total amount of 2.5 to 30 wt % based on the total weight of the antiperspirant cosmetic agent.

5. The antiperspirant cosmetic agent according to claim 1, wherein the at least one protein is included in a total amount of 3 to 20 wt % based on the total weight of the antiperspirant cosmetic agent.

6. The antiperspirant cosmetic agent according to claim 1, wherein the at least one protein has an average molecular weight M_{w} of 150 to 100,000 Da.

7. The antiperspirant cosmetic agent according to claim 1, wherein the at least one protein has an average molecular weight M_{w} of 180 to 50,000 Da.

8. The antiperspirant cosmetic agent according to claim 1, wherein the at least one protein has an average molecular weight M_{w} of 250 to 8,000 Da.

9. The antiperspirant cosmetic agent according to claim 1, wherein the at least one protein has an average molecular weight M_{w} of 300 to 5,000 Da.

10. The antiperspirant cosmetic agent according to claim 1, wherein the at least one protein causes a change in the light absorption in the event of a pH value change of at least 0.5 in a pH range of pH 4.5 to pH 7.5 at a concentration of 0.001 to 10 wt % of protein, with respect to the total weight of the sample mixture used for pH measurement, and at a temperature of 20° C.

11. The antiperspirant cosmetic agent according to claim 1, wherein the at least one protein causes a change in the light absorption in the event of a pH value change of at least 0.5 in a pH range of pH 5.0 to pH 7.0 at a concentration of 0.001 to 10 wt % of protein, with respect to the total weight of the sample mixture used for pH measurement, and at a temperature of 20° C.

12. The antiperspirant cosmetic agent according to claim 1, wherein the pH value change is caused by the addition of hydrogencarbonates or carbonates.

13. The antiperspirant cosmetic agent according to claim 1, wherein the at least one protein is selected from the group consisting of: (i) unmodified proteins; (ii) hydrolyzed proteins; (iii) chemically modified proteins; (iv) physically modified proteins; (v) hydrolyzed unmodified proteins; (vi) hydrolyzed and chemically modified proteins; (vii) hydrolyzed and physically modified proteins; and (viii) mixtures thereof.

14. The antiperspirant cosmetic agent according to claim 13, wherein the protein is a cationically modified protein and the cationically modified protein includes one or more residues of the formula R¹—N⁺(CH₃)₂—CH₂—CH(OH)—CH₂—X—R, in which R¹ represents an alkyl group having 1 to 30 carbon atoms, an alkenyl group having 1 to 30 carbon atoms, a hydroxyalkyl group having 1 to 30 carbon atoms, a C₁₀₋₁₈ alkyl, or a C₁₀₋₁₈ alkenyl group, X represents O, N, or S, and R represents the protein residue.

15. The antiperspirant cosmetic agent according claim 1, wherein the at least one protein is a keratin isolated from sheep's wool or goat's wool, wherein the keratin is hydrolyzed and/or wherein the keratin includes at least one lauryldimonium hydroxypropyl group.

16. The antiperspirant cosmetic agent according to claim 15, wherein the at least one protein includes lysine at 1.0 to 2.5 mol %, histidine at 0.5 to 0.8 mol %, and arginine at 7.0 to 8.0 mol %, with the respect to the total amount of substance of all amino acids of the protein.

17. The antiperspirant cosmetic agent according to claim 15, wherein the at least one protein has a calcium content of 100 to 600 mg, a potassium content of 700 to 1,400 mg, a phosphorus content of 40 to 150 mg, and a sulfur content of 1,100 to 5,700 mg, with respect to 1 kg of the at least one protein.

18. A packaging unit (kit of parts), comprising—formulated separate from each other—
a) at least one first container (C1), containing a cosmetic agent (M1) comprising at least one antiperspirant active substance, and
b) at least one second container (C2), containing a cosmetic agent (M2) comprising at least one protein, wherein the at least one protein occurs in cutaneous appendages of mammals, birds, fish, insects, or crustaceans, wherein the at least one protein causes a change in the light absorption of 1 to 100% in the event of a pH value change of at least 0.5 in a pH range of pH 4.0 to pH 8.0 at a temperature of 20° C. to 40° C. and a concentration of the protein of 0.001 to 10 wt %, with respect to the total weight of the sample mixture used to determine the change in the light absorption, and wherein the cosmetic agent (M2) includes no aluminum and/or zirconium halides and/or hydroxyhalides.

19. An antiperspirant cosmetic agent, comprising:
a) at least one substance selected from the group of cosmetic oils that are liquid at 20° C. and 1,013 hPa, odorous substances, and waxes,
b) propellant in a total amount of 0 to 99 wt %, with respect to the total weight of the antiperspirant cosmetic agent, and
c) at least one keratin isolated from sheep's wool or goat's wool in a total amount of 0.1 to 70 wt %, with respect to the total weight of the antiperspirant cosmetic agent, wherein the keratin is hydrolyzed and/or wherein the keratin includes at least one lauryldimonium hydroxypropyl group,
wherein the antiperspirant cosmetic agent includes no aluminum and/or zirconium halides and/or hydroxyhalides.

20. A non-therapeutic cosmetic method for preventing and/or reducing the perspiration of the body, comprising: applying the antiperspirant cosmetic agent according to claim 1 to a skin surface, and leaving the cosmetic agent on the skin surface for at least 1 hour.
